(19) United States
(12) Patent Application Publication
(10) Pub. No.: US 2020/0170604 A1
(43) Pub. Date: Jun. 4, 2020

(54) CT Based Probabilistic Cancerous Bone Region Detection

(71) Applicant: Howmedica Osteonics Corp., Mahwah, NJ (US)

(72) Inventors: Gokce Yildirim, Weehawken, NJ (US); Mark Gruczynski, Kinnelon, NJ (US)

(21) Appl. No.: 16/695,662

(22) Filed: Nov. 26, 2019

Related U.S. Application Data
(60) Provisional application No. 62/775,007, filed on Dec. 4, 2018.

(51) Int. Cl. A61B 6/00; A61B 6/03; A61B 34/20; A61B 34/00; G06T 7/00; G06T 7/55; G06T 7/73

(52) U.S. Cl. CPC A61B 6/505; A61B 6/032; A61B 34/20; A61B 34/76; G06T 7/0014; G06T 2207/30008; G06T 7/74; A61B 2034/2055; A61B 2034/2072; A61B 2034/2065; G06T 7/55

(57) ABSTRACT

A method of determining a boundary of a cancer of a bone of a patient includes imaging the patient's bone. A bone density ratio of interest may be obtained from the image of the bone, the bone density ratio of interest being a ratio of a first density of the bone at a first location in the image to a second density of the bone at a second location in the image. The obtained bone density ratio of interest may be compared to a reference bone density ratio of interest of a reference bone without bone cancer. Based on the comparison, it may be determined whether the cancer of the bone of the patient is present at the first location in the image or the second location in the image. The imaging may be CT imaging, and the imaging may include a first plurality of images in a first plane.

### CROSS-REFERENCE TO RELATED APPLICATIONS

This application claims the benefit of the filing date of U.S. Provisional Patent Application No. 62/775,007 filed Dec. 4, 2018, the disclosure of which is hereby incorporated by reference herein.

### BACKGROUND OF THE DISCLOSURE

When a patient presents with a bone tumor that has been positively identified as cancerous tissue, surgery may be an option to remove the portion of the bone that includes the cancerous cells in order to prevent further spread of the disease. In such a surgical procedure, it is typically important to correctly identify the cancerous portion of the bone to ensure that all of the cancerous bone is removed, preferably with enough accuracy to minimize the amount of healthy bone removed during the surgery. Often, medical imaging such as a PET scan is used in concert with radio-isotopic dyes to help identify the cancerous region. The results of such a PET scan, coupled with the surgeon's experience and intuition, are generally used in order to try to best remove all of the cancerous bone while removing the least amount of healthy bone. Recently, more accurate methods of removing cancerous bone tissue have been provided. For example, U.S. Patent Publication No. 2017/0181755, the disclosure of which is hereby incorporated by reference herein, describes the use of a robotic cutting tool to precisely remove cancerous cells from a bone. However, the robotic cutting tool can typically only be as accurate as the information input into the system regarding the actual boundaries of the cancerous cells.

The current technique of using radio-isotopic dyes injected into the patient's blood stream to attach the dye to the cancerous areas to detect the areas via a PET scan is slow. Further, if a custom implant is going to be created to replace the areas of bone removed during the surgical procedure, a CT scan of the bone may be required in addition to the PET scan. It would be desirable to reduce the amount of time required to detect the cancerous cell boundaries, and it would be desirable for that detection to be more accurate and reproducible. It would further be desirable to reduce the reliance on intuition and experience of surgical professionals in determining where the cancerous cell boundaries are and when enough of the bone has been removed during a surgical procedure.

### BRIEF SUMMARY

According to a first aspect of the disclosure, a method of determining a boundary of a cancer of a bone of a patient includes imaging the bone of the patient. A bone density ratio of interest may be obtained from the image of the bone, the bone density ratio of interest being a ratio of a first density of the bone at a first location in the image to a second density of the bone at a second location in the image. The obtained bone density ratio of interest may be compared to a reference bone density ratio of interest of a reference bone without bone cancer. Based on the comparison, it may be determined whether the cancer of the bone of the patient is present at the first location in the image or the second location in the image. The imaging may be CT imaging, and the imaging may include a first plurality of images in a first plane.

The obtaining step, comparing step, and determining step may be performed for each of the first plurality of images in the first plane. The imaging may include a second plurality of images in a second plane, and a third plurality of images in a third plane. The first plane, the second plane, and the third plane may be different planes. The first plane may be an axial plane, the second plane may be a sagittal plane, and the third plane may be a coronal plane. The obtaining step, comparing step, and determining step may be performed for each of the second plurality of images in the second plane and for each of the third plurality of images in the third plane. A three dimensional shape of the cancer may be defined based on the determining steps performed on each of the first, second, and third pluralities of images.

The reference bone density ratio of interest may be a reference ratio of a first reference density of a reference bone at a first reference location in a reference image to a second reference density of the reference bone at a second reference location in the reference image. The bone density ratio of interest may be based on a plurality of reference bones of a reference population of reference patients. The first location and the second location may be measured from an anatomical landmark. The first reference location and the second location may be measured from a reference anatomical landmark. The first and second locations, and the first and second reference locations, may be measured as percentile distances from the anatomical landmark and the reference anatomical landmark, respectively. The reference population may comprise a group of individuals having a parameter in common with the patient. The parameter may be selected from the group consisting of sex, age, and race.

The first density of the bone may be measured as a first value in Hounsfield units and the second density of the bone may be measured as a second value in Hounsfield units. The first and second locations may both be within a cortical shell of the bone. The first bone density may represent a first maximum bone density at the first location. The second bone density may represent a second maximum bone density at the second location.

### BRIEF DESCRIPTION OF THE DRAWINGS

FIG. 1 is a diagrammatic illustration of an exemplary operating room in which a haptic device is used with a computer-assisted surgery system.

FIG. 2 is a flowchart of a surgical method according to one aspect of the disclosure.

FIG. 3A illustrates an image of a bone to be treated by a surgical procedure according to one aspect of the disclosure.

FIG. 3B illustrates an aspect of a surgical plan for the bone of FIG. 3A.

FIG. 3C is a highly schematic representation of the haptic device of FIG. 1 performing a resection on the bone of FIG. 3A.

FIG. 3D is a highly schematic representation of the haptic device of FIG. 3C replacing the bone resected in FIG. 3A.

FIG. 3E is a highly schematic representation of the haptic device of FIG. 3C replacing the bone resected in FIG. 3A according to another aspect of the disclosure.

FIG. 4A is an illustration of the bone and bone tumor of FIG. 3A.

FIG. 4B is a representation of multiple axial slices of a CT scan on the bone of FIG. 4A.

FIG. 4C is a representation of density measurements taken at one of the axial slices shown in FIG. 4B.

### DETAILED DESCRIPTION

Prior to describing certain methods of detecting boundaries of cancerous cells, for example cancerous bone cells, descriptions of certain robotic surgical systems and methods that may be used to assist in removing such cancerous cells, once detected, are described.

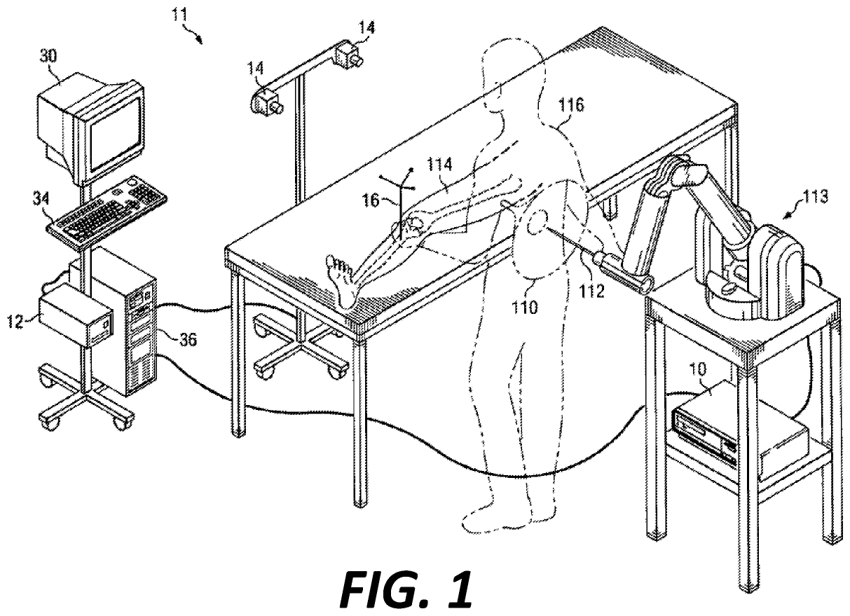

FIG. 1 is a diagrammatic illustration of an exemplary operating room in which a haptic device 113 is used with a computer-assisted surgery system 11. Computer-assisted surgery system 11 may include a display device 30, an input device 34, and a processor based system 36, for example a computer. Input device 34 may be any suitable input device including, for example, a keyboard, a mouse, or a touch screen. Display device 30 may be any suitable device for displaying two-dimensional and/or three-dimensional images, for example a monitor or a projector. If desired, display device 30 may be a touch screen and be used as an input device. One example of a system incorporating a haptic device 113 is described in greater detail in U.S. Pat. No. 7,831,292, the disclosure of which is hereby incorporated by reference herein.

Haptic device 113 is, in the illustrated example, a robotic device. Haptic device 113 may be controlled by a processor based system, for example a computer 10. Computer 10 may also include power amplification and input/output hardware. Haptic device 113 may communicate with computer-assisted surgery system 11 by any suitable communication mechanism, whether wired or wireless.

Also shown in FIG. 1 is a storage medium 12 coupled to processor based system 36. Storage medium 12 may accept a digital medium which stores software and/or other data. A surgical tool or instrument 112 is shown coupled to haptic device 113. Surgical tool 112 is preferably mechanically coupled to haptic device 113, such as by attaching or fastening it. However, if desired, surgical tool 112 may be coupled, either directly or indirectly, to haptic device 113 by any other suitable method, for example magnetically. Surgical tool 112 may be haptically controlled by a surgeon remotely or haptically controlled by a surgeon 116 present in proximity to surgical tool 112, although autonomous control with surgeon oversight is possible as well. Surgical tool 112 may be, for example, a bur, saw, laser, waterjet, cautery tool, or other trackable tool capable of cutting or otherwise shaping or resecting patent tissue, including bone. Patient tissue and bone may be referred to interchangeably herein and may include cartilage, tendons, skin tissue, and/or bone whether it be cortical or cancellous bone.

Haptic object 110 is a virtual object used to guide and/or constrain the movement and operations of surgical tool 112 to a target area inside a patient's anatomy 114, for example the patient's leg. In this example, haptic object 110 is used to aid the surgeon 116 to target and approach the intended anatomical site of the patient. Haptic feedback forces may be used to slow and/or stop the surgical tool's movement if it is detected that a portion of surgical tool 112 will intrude or cross over pre-defined boundaries of the haptic object. Furthermore, haptic feedback forces can also be used to attract (or repulse) surgical tool 112 toward (or away from) haptic object 110 and to (or away from) the target. If desired, surgeon 116 may be presented with a representation of the anatomy being operated on and/or a virtual representation of surgical tool 112 and/or haptic object 110 on display 30.

The computer-assisted surgery (“CAS”) system preferably includes a localization or tracking system that determines or tracks the position and/or orientation of various trackable objects, such as surgical instruments, tools, haptic devices, patients, donor tissue and/or the like. The tracking system may continuously determine, or track, the position of one or more trackable markers disposed on, incorporated into, or inherently a part of the trackable objects, with respect to a three-dimensional coordinate frame of reference. Markers can take several forms, including those that can be located using optical (or visual), magnetic or acoustical methods. Furthermore, at least in the case of optical or visual systems, location of an object's position may be based on intrinsic features, landmarks, shape, color, or other visual appearances, that, in effect, function as recognizable markers.

Any type of tracking system may be used, including optical, magnetic, and/or acoustic systems, which may or may not rely on markers. Many tracking systems are typically optical, functioning primarily in the infrared range. They may include a stationary stereo camera pair that is focused around the area of interest and sensitive to infrared radiation. Markers emit infrared radiation, either actively or passively. An example of an active marker is a light emitting diode (LED). An example of a passive marker is a reflective marker, such as ball-shaped marker with a surface that reflects incident infrared radiation. Passive systems may include an infrared radiation source to illuminate the area of focus. A magnetic system may have a stationary field generator that emits a magnetic field that is sensed by small coils integrated into the tracked tools.

With information from the tracking system on the location of the trackable markers, CAS system 11 may be programmed to be able to determine the three-dimensional coordinates of an end point or tip of a tool and, optionally, its primary axis using predefined or known (e.g. from calibration) geometrical relationships between trackable markers on the tool and the end point and/or axis of the tool. A patient, or portions of the patient's anatomy, can also be tracked by attachment of arrays of trackable markers. In the illustrated example, the localizer is an optical tracking system that comprises one or more cameras 14 that preferably track a probe 16. As shown in FIG. 1, cameras 14 may be coupled to processor based system 36. If desired, cameras 14 may be coupled to computer 10. Probe 16 may be a conventional probe. If desired, the probe may be rigidly attached to haptic device 113 or integrated into the design of haptic device 113.

In one implementation, processor based system 36 may include image guided surgery software to provide certain user functionality, e.g., retrieval of previously saved surgical information, preoperative surgical planning, determining the position of the tip and axis of instruments, registering a patient and preoperative and/or intraoperative diagnostic image datasets to the coordinate system of the tracking system, etc. Full user functionality may be enabled by providing the proper digital medium to storage medium 12 coupled to computer 36. The digital medium may include an application specific software module. The digital medium may also include descriptive information concerning the surgical tools and other accessories. The application specific software module may be used to assist a surgeon with planning and/or navigation during specific types of procedures. For example, the software module may display predefined pages or images corresponding to specific steps or stages of a surgical procedure. At a particular stage or part of a module, a surgeon may be automatically prompted to perform certain tasks or to define or enter specific data that will permit, for example, the module to determine and display appropriate placement and alignment of instrumentation or implants or provide feedback to the surgeon. Other pages may be set up to display diagnostic images for navigation and to provide certain data that is calculated by the system for feedback to the surgeon. Instead of or in addition to using visual means, the CAS system could also communicate information in other ways, including audibly (e.g. using voice synthesis) and tactilely, such as by using a haptic interface. For example, in addition to indicating visually a trajectory for a drill or saw on the screen, a CAS system may feed information back to a surgeon whether he is nearing some object or is on course with an audible sound. To further reduce the burden on the surgeon, the module may automatically detect the stage of the procedure by recognizing the instrument picked up by a surgeon and move immediately to the part of the program in which that tool is used.

The software which resides on computer 36, alone or in conjunction with the software on the digital medium, may process electronic medical diagnostic images, register the acquired images to the patient's anatomy, and/or register the acquired images to any other acquired imaging modalities, e.g., fluoroscopy to CT, MRI, etc. If desired, the image datasets may be time variant, i.e. image datasets taken at different times may be used. Media storing the software module can be sold bundled with disposable instruments specifically intended for the procedure. Thus, the software module need not be distributed with the CAS system. Furthermore, the software module can be designed to work with specific tools and implants and distributed with those tools and implants. Moreover, CAS system can be used in some procedures without the diagnostic image datasets, with only the patient being registered. Thus, the CAS system need not support the use of diagnostic images in some applications—i.e. an imageless application.

Haptic device 113 may be used in combination with the tracking and imaging systems described above to perform highly accurate bone resections and grafting bone on the resected bone. A general description of such a procedure is described below, followed by at least one example of a method to determine the boundaries of cancerous bone to be removed using a surgical robotic system. However, it should be understood that the method(s) to determine the boundaries of cancerous bone could be used without a corresponding robotic surgical procedure. In other words, once the cancerous bone boundaries are detected, the cancerous bone may be removed in any desired fashion, although robot or robot-assisted surgical procedures may be preferred to increase the accuracy of the surgical procedure.

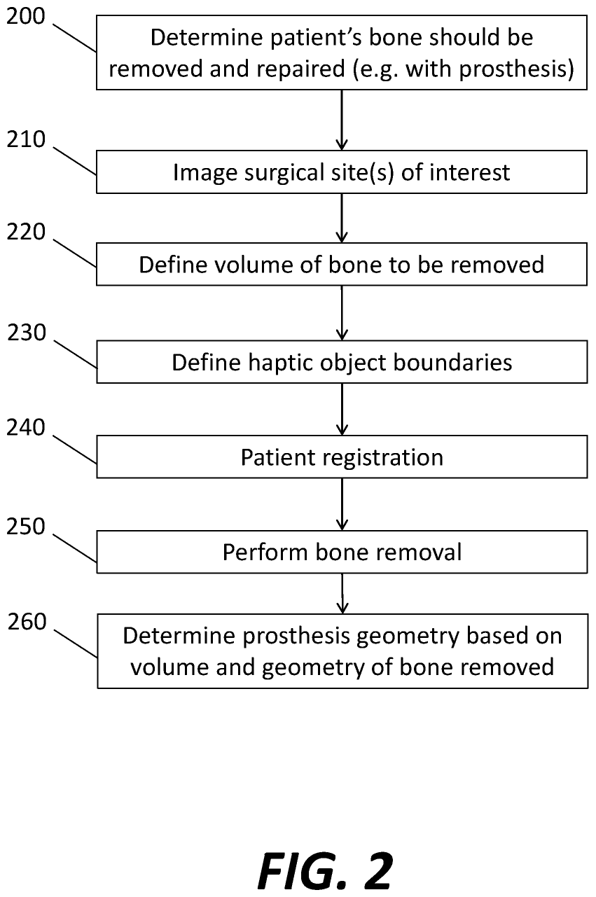

FIG. 2 illustrates a flow chart of a surgical procedure according to the present disclosure. In a first step 200, a physician or other medical practitioner diagnoses that a patient would benefit from having a portion of a bone removed or resected followed by implantation of a prosthesis onto the bone at or near the site of resection. In this regard, the term prosthesis encompasses transplanted bone including, for example, allograft, autograft, xenograft, or bone substitute as well as other biologics, metals, plastics, and combinations thereof. It should be understood that, although step 200 is shown as a separate step, the actual determination that a portion of a patient's bone should be removed need not be a separate step. In other words, the methods of detecting boundaries of bone cancer described herein may actually result in the diagnosis and the determination that a portion of the patient's bone should be removed. Further, although upon removal of a portion of the patient's bone, a prosthesis would typically be implanted in place of the removed bone, in some instances there may not need to be a separate prosthesis implanted onto the bone once the cancerous bone is detected and removed.

After determining the intended surgical site, the surgical site may be imaged in step 210, for example via an MRI or CT scan, or any other suitable imaging modality. The images may be uploaded or otherwise transferred to processor based system 36 for use on the software residing therein. Three-dimensional models of individual bones and/or joints may be created from the images taken of the surgical site. Systems and method for image segmentation in generating computer models of a joint to undergo arthroplasty is disclosed in U.S. Pat. No. 8,617,171, the disclosure of which is hereby incorporated by reference herein. The images may be processed or otherwise used in order to plan portions of the surgical procedure in step 220. In one example, the desired geometry and/or volume of the bone to be removed or resected may be defined based on the images. The surgeon may define the geometry and/or volume using the software with manual definition or semi-automatic definition. For example, the surgeon may outline geometric boundaries on the images on display 30 with input device 34, such as a mouse, to determine the geometry and/or volume of bone to be removed. In addition or alternatively, the software may employ image processing to identify damaged areas of the bone, for example by determining bone quality, for example by analyzing bone density based on brightness or other parameters of the image, to provide for a suggested geometry and/or volume of bone removal which may be confirmed or altered by the surgeon. It should be understood that this geometry and/or volume definition step 220 may be performed prior to the surgical procedure on a separate computer system, with the results of this step imported to processor based system 36. It should also be understood that the steps shown in FIG. 2 do not necessarily need to be completed in the order shown. For example, a patient may be first imaged in step 210, and based on the results and analysis of the imaging, the determination that surgical intervention is required in step 200 may be made.

In step 230, the surgeon may define the boundaries of haptic object 110. This may be accomplished in one of several ways. In one example, the haptic object 110 may be based on the geometry and/or volume of bone to be removed determined in step 220. The haptic object 110 may be defined to have boundaries along the geometry and/or volume of bone to be removed so that the surgical tool 112, as described above, may aid the surgeon 116 to target and approach the intended anatomical site of the patient with surgical tool 112. In another example, a number of pre-defined shapes or volumes may be pre-loaded into computer 10 and/or computer 36. For example, different procedures may have certain typical shapes or volumes of intended bone removal, and one or more pre-loaded geometries and/or volumes may be included in the software application on computer 10 and/or computer 36, for example with each geometry and/or volume corresponding to one or more types of procedures. These pre-loaded shapes or volumes may be used without modification, but in many cases the pre-loaded geometries and/or volumes will be modified by the surgeon and/or combined with other pre-loaded geometries and/or volumes to meet the needs of the particular patient.

In step 240, haptic device 113 is registered to the anatomy of the patient. If desired, a representation of the anatomy of the patient displayed on display device 30 may also be registered with the anatomy of the patient so that information in diagnostic or planning datasets may be correlated to locations in physical space. For example, the haptic device 113 (or a probe attached thereto) may be directed to touch fiducial markers screwed into the bones, to touch a series of points on the bone to define a surface, and/or to touch anatomical landmarks. The registration step 240 is preferably performed when the anatomy is clamped or otherwise secured from undesired movement. Registration may also be performed using, for example, intraoperative imaging systems. However, the anatomy does not need to be clamped in certain situations, for example if tracking devices are coupled to the anatomy. In that case, any movement of the anatomy is tracked so that rigid fixation is not necessary.

In step 250, with patient registration complete, the bone removal procedure is performed. The procedure may be any suitable procedure in which bone is to be removed, such as resection in preparation for joint replacement, bulk bone removal, or small volume bone removal for treating small tumors or the like. The actual process of removing bone may be performed semi-autonomously under haptic control, as described above, autonomously by haptic device 113, manually via free-hand resection by the surgeon, or any combination of the above. Regardless of the specific procedure or the level of surgeon control, the bone removal geometry and/or volume is tracked by computer 10 (and/or computer 36) by tracking the position of surgical tool 112 with the navigation system and/or joint encoders of haptic device 113. Thus, even if the bone actually removed differs from the surgical plan, the computer 10 (and/or computer 36) tracks and stores information relating to the bone actually removed. In other embodiments, photo and/or pressure sensors may be employed with haptic device 113 to precisely measure the geometry and/or volume of bone that is removed. It is also contemplated that, following the bone removal, additional imaging may be performed and compared to patient images prior to the resection to determine bone actually removed, which may be used as an alternative to the robotic tracking of bone removal or as confirmation of same. Still further, instead of tracking and storing information to the bone actually removed during the removal process, the bone may first be removed, and following the bone removal, the remaining surface of the bone may be probed to register the precise remaining volume and/or geometry of bone. And it should be understand that, as noted above, in some circumstances it is conceivable that, following bone removal, an implant is not needed to replace the removed bone or to otherwise stabilize or secure the remaining bone. In such scenarios, it may not be necessary to track the removal of the bone.

With the information relating to the geometry and/or volume of bone removed from the patient, computer 10 and/or computer 36 determines the precise three-dimensional geometry of the prosthesis to be implanted into or onto the bone in step 260. Based on this determination, haptic device 113 may be used in any one of a number of ways to form and/or place the prosthesis. For example, if the prosthesis is an allograft bone, haptic device 113 may employ the determined geometry and/or volume to assist the surgeon in shaping the allograft bone to precisely fit the geometry of the resected bone. Alternatively, a similar procedure may be used on the patient if the prosthesis is an autograft bone taken from another bone portion of the patient, with the haptic device 113 providing assistance to the surgeon in resecting the precise geometry and/or volume of autograft to replace the bone removed in step 250. In other embodiments, haptic device 113 may be employed to resect more autograft than will be needed to replace the bone removed in step 250 while taking into account whether such removal of autograft taken from the other bone portion of the patient is safe for the patient. Still further, a liquid or putty-type bone graft may be applied to the site of bone removal in step 250, for example by attaching a syringe-like device as the tool of haptic device 113, with precise application of the bone graft to the site of bone removal. Some of these examples are described in greater detail below.

As noted above, steps 200 through 260 do not necessarily need to be performed in the order shown in FIG. 2, nor do all the steps need to be performed in a given procedure, and, as noted above, some steps may be combined into a single step. For example, in some cases, it may be preferable to prepare the prosthesis prior to resecting the patient's bone. This may be true in the case of an autograft prosthesis since the donor tissue maybe limited and/or difficult to access. In such a case, the autograft may be prepared according to the surgeon's experience (manually or otherwise), the intended surgical procedure, and/or any pre- and intra-operative planning Once the prosthesis is formed, the prosthesis may be probed and registered to using computer 10 and/or computer 36 so that the volume and/or geometry of the prosthesis is stored. The volume and/or geometry of the prosthesis may then be used to create the haptic object 110, so that the surgeon may use the haptic device 113 to resect the patient's bone to a shape corresponding to the geometry and/or volume of the previously prepared prosthesis.

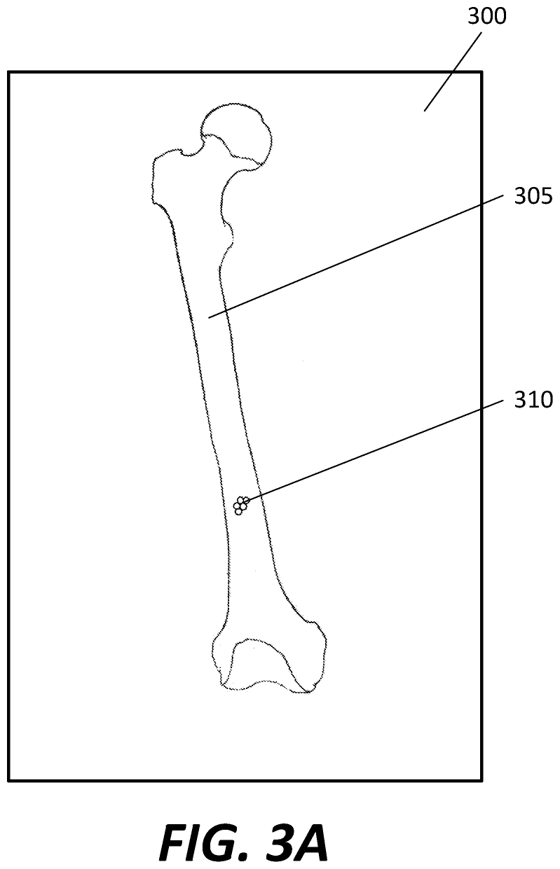

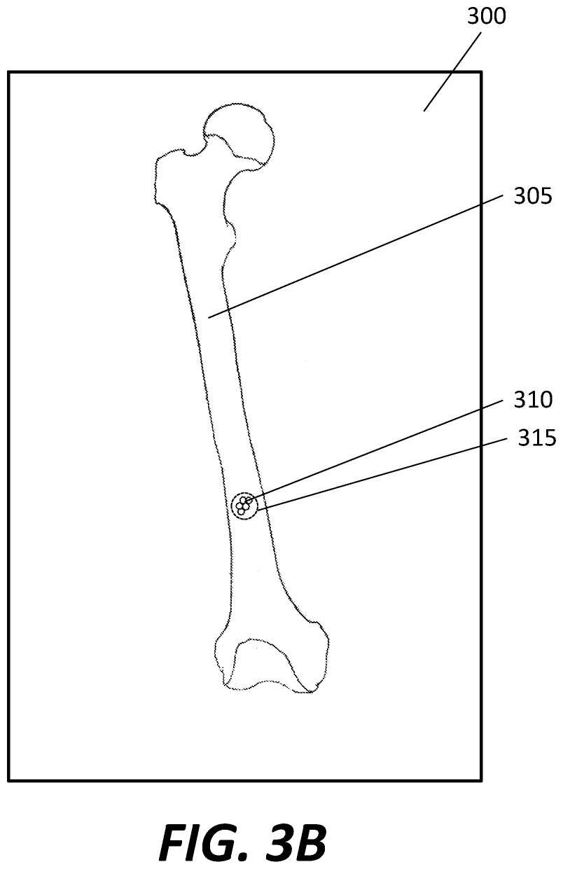

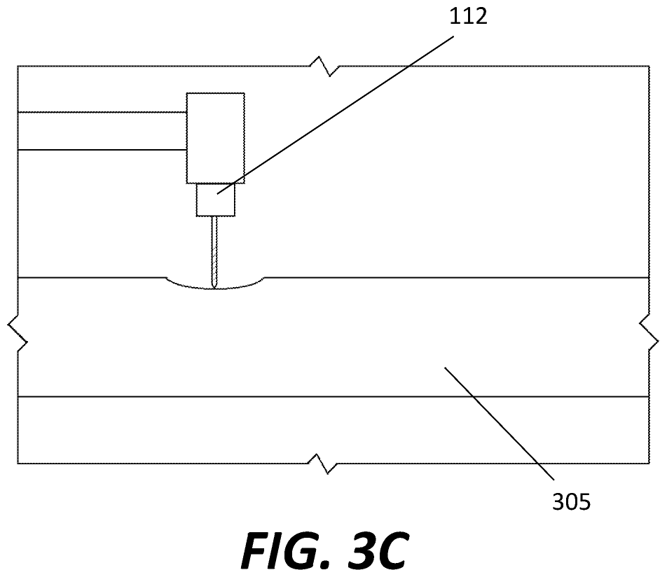

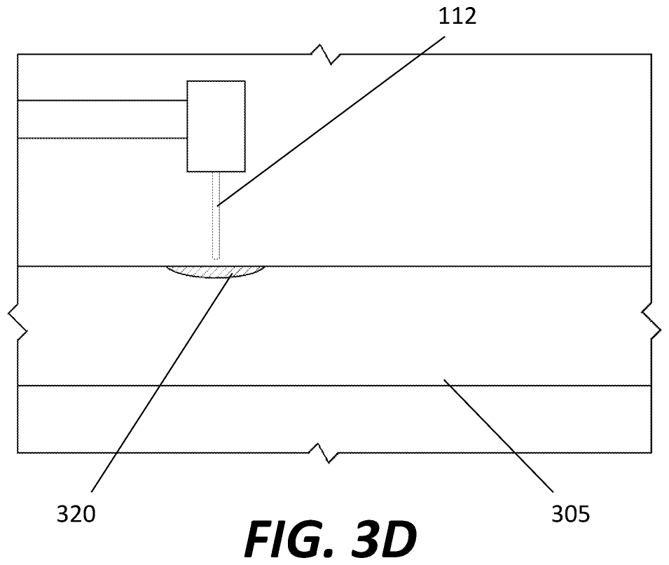

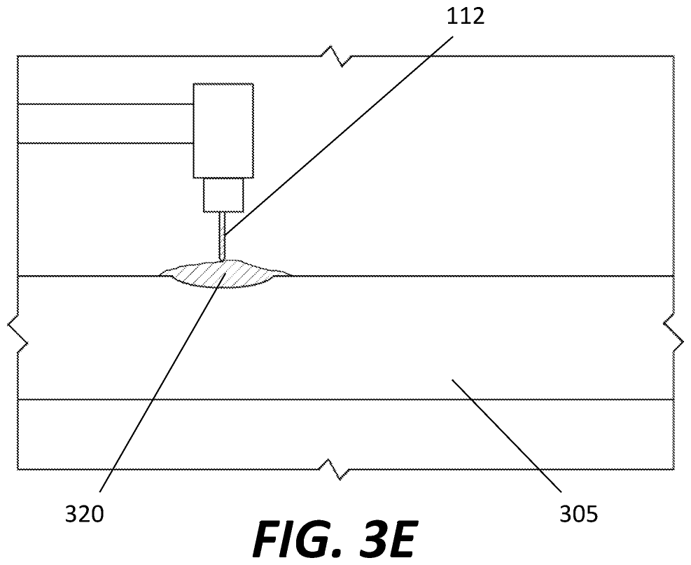

One particular example of a procedure utilizing one or more of steps 200 through 260 of FIG. 2 is for treating bone tumors. Common types bone tumors that may be treated according to the below procedure may include giant cell tumors of bone, benign aneurysmal bone cysts, and malignant low grade chondrosarcomas. The patient's bone, including the tumor site, is imaged in step 210. A highly schematic illustration of an image 300 of a patient's femur 305 is shown in FIG. 3A with a bone tumor(s) 310 shown on the image. The image 300, or a set of images 300, may be uploaded or otherwise stored on processor-based system 36.

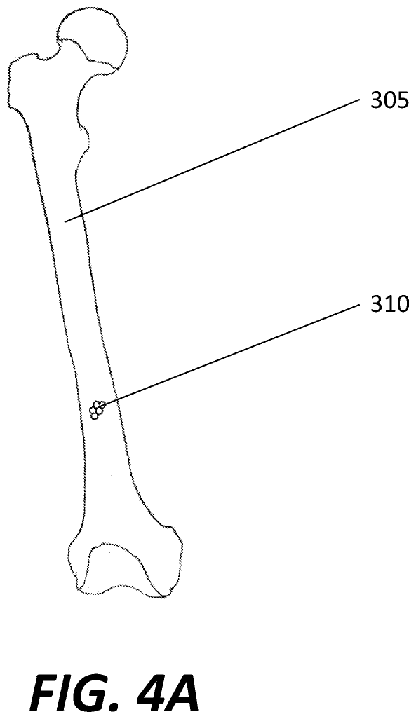

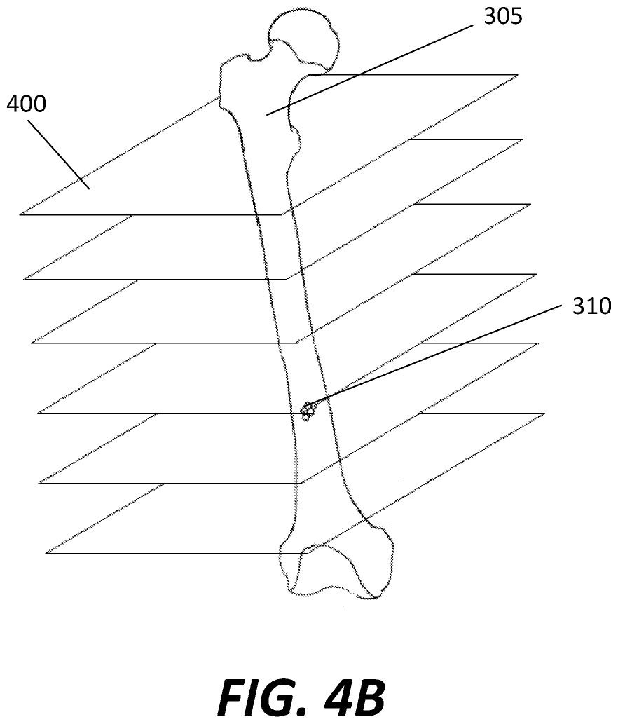

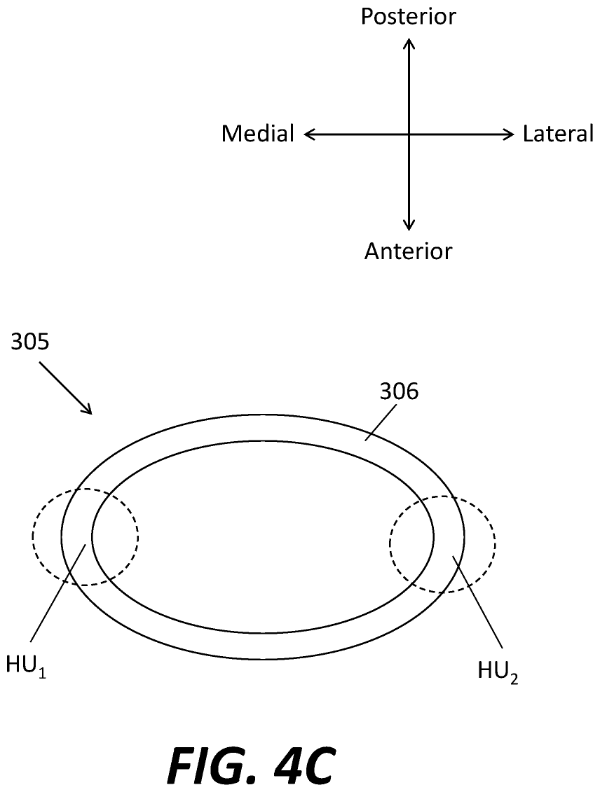

The processor-based system 36, for example with the aid of software, may automatically identify the location and/or boundaries of tumors(s) 310. In one example, this determination is based on bone density and/or quality information from the image 300. Tumor(s) 310 and surrounding portions of healthy femur 305 may have different density values, allowing for the correlation of image brightness to bone density in order to determine the boundaries between tumor(s) 310 and adjacent portions of healthy femur 305. The surgeon may review and confirm the determined location of tumor(s) 310, revise the determined location of the tumor(s), or otherwise manually identify the location of the tumor(s). Additional details regarding the determination of the cancerous cells are described below in connection with FIGS. 4A-C.

Based on the determination of the boundary between tumor(s) 310 and healthy femur 305, the processor-based system 36 may automatically determine the geometry and/or volume 315 of femur 305 to be resected to effectively remove tumor(s) 310, as provided by step 220 and as shown in FIG. 3B. In one example, the processor-based system 36 may apply a three-dimensional buffer around the determined boundary between tumor(s) 310 and healthy femur 305, for example a buffer of 0.5 mm, 1 mm, 2 mm, or 3 mm outside the boundary to help ensure that the removal of tumor(s) 310 is complete. In other examples, the software-based system 36 may provide a standard buffer, for example 1 mm, and the surgeon may confirm the buffer or revise the buffer. Still further, the surgeon may manually input the geometry and/or volume of bone to be removed, using his or her discretion regarding any appropriate buffer beyond the determined location of tumor(s) 310. Based on the geometry and/or volume 315 of bone to be removed, the system may determine a haptic object 110 correlating to the geometry and/or volume 315 as provided in step 230. As described in greater detail below, it is also contemplated that the surgeon may skip the step of defining the volume of bone to be removed, rather using his or her own experience to resect the bone to remove tumor(s) 310 using haptic device 113. As is described in greater detail below, the resection may alternately be a manual resection procedure.

Whether or not steps 220 and 230 are performed, the patient is then registered to the haptic device 113 as described above in connection with step 240. A surgical tool 112 in the form of a small bur may be coupled to haptic device 113 and used to remove the tumor(s) 310 on femur 305. If steps 220 and 230 were performed, the haptic device 113 may autonomously or semi-autonomously guide the bur using the constraints of the haptic object 110 to remove the desired geometry and/or volume 315 of bone, as shown in FIG. 3C. If steps 220 and 230 were not performed, the surgeon may manually guide the bur through manipulation of the haptic device 113. In either scenario, the path of the bur is tracked and information regarding the actual volume of bone removed is stored in computer 10 (and/or computer 36). Preferably, the tip and/or sides of the bur, or any relevant cutting surfaces, are tracked. It is further contemplated that, if steps 220 and 230 are not performed, a manual device, such as a curette, may be employed by the surgeon to remove the tumor(s) 310. The curette may be provided with a tracking array and be operatively coupled to computer 10 (and/or computer 36) so that the movements of the curette in space relative to the patient's bone are tracked, so that the precise volume of bone removed may be tracked for use in replacing the removed bone. For each example above, because the three-dimensional position of the patient's bone is known via registration and the image(s) 300, and the three-dimensional position of the surgical tool (e.g. bur or curette) is known via the tracking system, any time the tip of the surgical tool 112 intersects with the patient's bone, the portion of bone removed may be identified and stored by computer 10 (and/or computer 36).

In step 260, the precise geometry and/or volume of the prosthetic is determined. The prosthetic geometry and/or volume may be identical to that of the bone removed, as tracked during the removal step, whether the bone removal was autonomous, semi-autonomous, or manual. If the bone removal geometry and/or volume was pre-planned using computer 36, the geometry and/or volume of the prosthetic may be identical to the geometry and/or volume of the planned bone removal, since haptic device 113 helps ensure the bone removal occurs exactly (or nearly exactly) according to plan. Instead of forming the geometry of the prosthesis to be identical to the geometry and/or volume of the removed bone, modifications may be made, for example so that the prosthesis can have a press fit or interference fit with the patient's anatomy.

The prosthesis may take any suitable form, including, e.g., demineralized bone matrices (“DBM”), morselized autograft, morselized allograft, polymethyl methacrylate (“PMMA”) bone cements, synthetic calcium phosphate or calcium sulfate based bone grafts, and/or ultraviolet (“UV”) curable resins. If the prosthesis takes the form of one of the above void fillers, it may be delivered via syringe or syringe-like device. For example, as shown in FIG. 3D, the haptic device 113 may include a surgical tool 112 in the form of a syringe-like device packed with void filler 320. The void filler 320 may be ejected from the end effector 112 by haptic device 113 to precisely fill the volume of bone previously removed with the void filler 320. Alternatively, the void filler 320 may be deposited in some other desired geometry and/or volume within the resected bone, such as a partial fill.

Rather than use a homogenous void filler 320, the process may be divided into steps to provide additional features of the prosthetic bone. For example, a surgical tool 112 with a syringe packed with a curable resin, such as a UV curable resin, may be coupled to haptic device 113. A curing source, such as a UV source, may be provided along with surgical tool 112 so that the curable resin cures contemporaneously or near-contemporaneously upon deposition into the bone void. A cured resin lattice may be formed in this manner, which may be then be infused with a void filler or a bone growth composition. The lattice may take the form of a structural three-dimensional matrix with voids that can be filled with a void filler and/or bone growth composition. This infusion may be accomplished by coupling a surgical tool 112 in the form of a syringe-like device packed with the bone growth material to haptic device 113, or manually by the surgeon.

Another alternative, as shown in FIG. 3E, is to apply a large mass of void filler 320 into the void, for example manually, to partially or completely fill the void. If the void is completely filled with void filler 320, a bur or other surgical tool 112 is coupled to haptic device 113, and the haptic device 113 may autonomously or semi-autonomously cut away extraneous void filler 320 until the remaining void filler exactly matches the geometry and/or volume of resected bone.

With any of the void filler 320 deposition techniques described above, the void filler 320 may vary in quality in three-dimensions. For example, layers of filler 320 which have different densities may be applied as desired, for example by repeating the delivery described in connection with FIG. 3D in sequential steps using different fillers with different densities. This method may facilitate more closely mimicking the natural bone, for example where inner layers of cancellous bone are less dense than outer layers of cortical bone. Other ways to achieve variable prosthesis properties such as variable density include, for example, adding beads, mesh materials, or fibrous materials to the filler material. Still further, different layers may be deposited in an alternating fashion, such as a hard prosthesis having a liquid or filler material underneath and also on top of the hard prosthesis.

Some void fillers 320, such as bone cement, may be applied to the bone at a relatively high temperature and cure as the cement cools. The surgical tool 112 may incorporate a thermal sensor so that computer 10 (and/or computer 36) is able to detect a temperature of the void filler 320 packed into the effector. The computer 10 (and/or computer 36) may then control the deposition of the void filler 320 onto the bone so that the application occurs at an optimal viscosity and/or thermal optimum. For example, if the void filler 320 is too hot, the native bone may be damaged. However, if the void filler 320 is allowed to cool too much prior to deposition, the deposition may not be effective if the void filler 320 has already begun to harden.

Although the procedure above is described as tracking bone removal coincident with the bone removal process, other alternatives may be suitable. For example, after the bone removal is complete, a shapeable material may be pressed into the bone void to create a mold having a volume and/or geometry corresponding to the resected bone. It should be understood that this mold may actually be a “reverse” mold of the resected bone, since the mold has the shape of what was removed. The mold, once formed, may be removed from the bone and the surface probed and registered to determine the shape of the removed bone (and correspondingly the shape of the remaining bone).

As noted above, generally, the more accurate the determination of the boundaries of cancerous bone cells is, the more accurate the removal of those cancerous cells can be, along with a corresponding decrease in the amount of healthy bone that needs to be removed to ensure that the cancerous cells are fully excised. Although PET scans may be suitable in some instances, better methods of detecting the boundaries of the cancerous tissue may be desirable. Further, although the above description includes an indication that bone tumors 310 on femur 305 may have a different density than healthy portions of the femur, additional information may be utilized to more accurately determine the boundaries of the cancerous cells, including in two and preferably three dimensions. And, while the description below is provided in the context of femur 305, it should be understood that the description may apply with equal force to any bone in the body, as well as any other tissue that can be tracked using imaging modalities.

FIG. 4A illustrates femur 305 including bone tumors 310, similar to that shown in FIG. 3A. Although an X-ray and/or CT image or set of images may allow the bone tumors 310 to be seen visually, the exact boundaries of the tumors may be much more difficult to determine. One solution to the problem of determination of the boundaries of the cancerous cells is by determining ratios of bone densities at different points along the bones for healthy populations, and comparing the same ratios for the particular patient to the expected healthy ratios.

In the example of a femur 305 with a bone tumor 310, a CT scan can be performed on the femur 305 to create a plurality of image scans or “slices” 400, as shown in FIG. 4B. In the example of FIG. 4B, the slices 400 are taken axially along an axis of the femur 305. Typically, high resolution imaging is desirable in order to obtain a relatively large amount of information. In this particular example, the imaging is performed along the anatomical axis of the femur 305. By performing the imaging along the anatomical axis of the femur 305, the results of the imaging, as described in greater detail below, may more easily be compared to data obtained from imaging along the anatomical axis of femurs of a healthy population. In other words, using imaging along anatomical axes, as opposed to a different axis such as the mechanical axis, may reduce or eliminate complications from variations between individual patients in terms of how the bone is angled with respect to other anatomy. And while the example below is further described with the example of axial slices 400, it should be understood that the scan can be performed in three dimensions, including for example coronal and sagittal planes, to create three-dimensional information regarding the three-dimensional boundaries of the tumor 310.

FIG. 4C illustrates an exemplary slice 400 of the scan shown in FIG. 4B. For the axial slice example, certain ratios of bone density (as measured, for example, in Hounsfield units) of the femur 305 may be determined. For example, portions of the cortical shell 306 of the femur 305 may be analyzed to determine bone densities as expressed in Hounsfield units. In one example, the greatest density HU₁ of the medial cortical bone 306 in the particular slice may be compared to the greatest density HU₂ of the lateral cortical bone 306 in the particular slice to determine the ratio of interest

$$\frac{{HU}_{1}}{{HU}_{2}} .$$

It should be understood that although this disclosure describes determining density and density ratios, the actual bone density need not be determined. For example, by using a ratio of Hounsfield units, which may relate to density (e.g. denser bone generally presents as brighter pixels in a CT image versus less dense bone presenting as darker pixels in the image), imaging conditions may become less important when comparing the ratios of interest from one patient to another. In other words, imaging conditions and procedures could result in bone having the same density in the two scans appearing with different brightness, despite the density value being the same or near identical. By utilizing ratios of different Hounsfield units within the same scan, the effects of the imaging conditions may be reduced or eliminated.

A ratio of interest

$$\frac{{HU}_{1}}{{HU}_{2}}$$

may be determined for each slice 400 in the scan of the patient's femur 305 to determine a particular density ratio profile. This information may be compared to profiles of known patients with healthy (e.g. non-cancerous) bones. For example, density ratio profiles may be determined for a plurality of patients with healthy, non-cancerous femurs. This information may be acquired from any suitable source, including, for example, the Stryker Orthopaedics Modeling and Analytics system (“SOMA”) database. The healthy bone data may be used to create a profile for comparison to the patient's data to determine where along the femur 305 the patient's ratios of interest deviate from the expected ratios of interest of a patient with a healthy, non-cancerous femur in order to identify the boundaries of the cancerous bone. The data of the patients with the healthy femurs may be grouped with certain sub-classes, for example based on age range, ethnicity, and sex. In other words, if the patient with the bone tumor 310 is a middle-aged Caucasian male, the ratios of interest

$$\frac{{HU}_{1}}{{HU}_{2}}$$

of the patient may be compared to the ratios of interest

$$\frac{{HU}_{1}}{{HU}_{2}}$$

of other middle-aged Caucasian males with non-cancerous femurs, although it should be understood that other sub-groups of combinations of sub-groups may be used, if desired. Sub-group tendencies may be determined, for example, on regression analyses.

Still referring to the exemplary axial scan of a patient's femur 305, it should be understood that the comparison between the patient's bone density ratios to healthy bone density ratios may be controlled or normalized in a variety of ways. First, as already noted above, by using ratios of Hounsfield units instead of simply comparing Hounsfield units, differences in imaging conditions and/or imaging protocols may be reduced or eliminated. Second, in the example in which the bone is a femur 305 and the scans are taken using a plurality of axial slices 400, the points of comparison along the patient's femur versus the healthy femur data may be based on percentile distances from common anatomic landmarks, which may help normalize for variations in patient sizes. For example, the axial slices may be taken at known percentile distances between the hip joint center and the knee joint center when the femur 305 is the bone of interest. Thus, for example, ratio of interest

$$\frac{{HU}_{1}}{{HU}_{2}}$$

as measured at the halfway point between the hip joint and the knee joint of the patient may be compared to known ratios of interest

$$\frac{{HU}_{1}}{{HU}_{2}}$$

at the halfway point between the hip joint and the knee joint of individuals with healthy, non-cancerous femurs. By normalizing the data in this fashion, the comparisons between the patient of interest and known healthy patient data are more relevant. It should be understood that for other bones, other relevant anatomical landmarks may be used for the same purpose of normalization.

For the illustrated example of axial slices 400 of a femur 305, it should be understood that the entire femur may not need to be scanned, and instead portions of the femur near the bone tumor 305 may be scanned and density ratios calculated and compared to healthy bones, depending on the particular case conditions. A large number of slices 400 may be taken at any desired resolution to provide as much information as desired for comparison. When comparing the patient's data to the data of non-cancerous bones, the slices 400 in which there is deviation from the healthy patient's data may be flagged as including the cancerous tissue, as the density of the cancerous tissue is expected to deviate from the density of healthy tissue. While the axial scan data may provide for information regarding boundaries of the bone tumor 310 in a single dimension, scans may also be taken in other planes, such as the sagittal and coronal planes and similarly compared to the same information determined from a database of healthy patient bones to create a three-dimensional perimeter of the patient's bone tumor 310. It should be understood that the process of determining density ratios of interest in other scanning planes may be essentially the same as described in connection with the axial scans 400. For example, if high resolution CT scans are performed in the axial, coronal, and sagittal planes and relevant ratios of interest compared to corresponding population data of healthy bones, a set of image slices of the patient may be marked as likely to indicate cancerous cells. The volume in which those marked slices coincide with one another may define the volume of the tumor 310. In other words, the ratio analysis may be performed in all three dimensions to determine the xyz coordinates of the ratio “cubes” that are out of range for the patient. That cluster of cubes that are out of range based on the xyz density (or Hounsfield unit) ratio calculations are deemed to be cancerous and should be removed, with those cube cluster boundaries able to be displayed to the surgeon to assist in the removal. It should be understood that various method of analyzing the data may be suitable, for example including nearest neighbor analysis, known as k-NN in data analytics.

Further, although one particular example of a density ratio of interest is provided above, other density ratios may be used in the alternative or in addition to that shown. For example, for axial scans 400, the maximum Hounsfield measurement at the anterior cortical shell 306 may be compared to the maximum Hounsfield measurement at the posterior cortical shell 306 to provide another ratio for comparison to healthy bones. Density ratios analyses of superior versus inferior bone may also be performed. By comparing scans in three planes of the patient's bone to the corresponding scans of a population of non-cancerous patients' bones, it can be determined, based on the shift in density ratios between the patient of interest and the healthy population, which image slices of the patient's bone are likely to contain the cancerous bone. Because the scans are taken in three planes, the data can be compiled to determine the three-dimensional perimeters of the cancerous bone to accurately and precisely determine the boundaries of the cancerous bone. That information can be entered into a computer system, for example the systems described above, so that a robotic surgical tool can be used to very precisely cut out the entire volume of cancerous bone without removing any (or removing only a small pre-determined buffer) of healthy bone stock.

It should be understood that although the bone density ratio profile of a particular patient may be manually (or autonomously) compared to one or more bone density profiles of other patients with corresponding healthy non-cancerous bones, an alternative is to create a statistical (or other) model in which bone density information of a particular patient may be input into the model, the model being based on information derived from the database of bone density profiles of other individuals, and the model may output the expected boundaries of the patient's bone tumor.

Referring back to FIG. 2, the above-described method of determining the boundary of a bone tumor in a patient may be performed as step 220. This may be performed as purely a diagnostic test, as part of a planned surgical procedure, or in preparation for a surgical procedure. For example, if a patient is expected to have a bone tumor, the relevant bone may be imaged in a CT scan as described above and the information from the CT scan regarding bone density ratios be compared to population (or relevant sub-population) data of corresponding non-cancerous bones to determine if a deviation in the bone density ratios indicates cancer. Whether this is performed manually, semi-automatically, or fully automatically, for example through the use of a statistical model or another algorithm, the imaging of the patient may be purely a diagnostic tool if desired. Whether used as a diagnostic tool or as part of a surgical procedure, the determined boundaries of the patient's bone tumor (with or without additional input and confirmation from a surgeon or other medical personnel) may be input into a computer system, such as that described above in connection with FIG. 1 in order to define the volume of bone that should be removed to fully remove the cancerous cells. Again, as noted above, a buffer area may be added in order to increase the comfort that all cancerous cells are, in fact, removed upon cutting the bone according to the determined boundaries of the cancerous cells. The procedure may be largely performed as described above in connection with FIG. 2, including the use of a robotic cutting tool to remove the bone. If a prosthesis is going to be implanted to replace the removed bone, the shape of the prosthesis may be determined as described above, for example by tracking the volume of the bone that the robot cuts, or otherwise may be based on the boundaries of the cancerous cells determined during the imagining analysis.

Some of the benefits of using the above-described method to determine the boundaries of bone cancer include increased accuracy and a reduction in the necessity for subjective analysis by a surgeon, which may in turn reduce variability in results. Further, the information of the boundaries of the bone cancer may have additional use, not only for diagnostic purposes, but also in determining how to create a prosthesis having the appropriate fit to replace the cancerous bone once it is removed. Still further, in many scenarios the patient is likely to require CT imaging for other purposes of surgical planning, so the method of determining the boundaries of the tumor using CT imagining and related analysis may not require the patient to undergo additional procedures. For example, it may be possible to fully eliminate the need for a PET scan to determine the boundaries of the bone tumor, where traditionally a PET scan and CT imaging may both be performed in preparation for surgery to remove cancerous bone cells.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of determining a boundary of a cancer of a bone of a patient, the method comprising:
imaging the bone of the patient;
obtaining from the image of the bone a bone density ratio of interest, the bone density ratio of interest being a ratio of a first density of the bone at a first location in the image to a second density of the bone at a second location in the image;
comparing the obtained bone density ratio of interest to a reference bone density ratio of interest of a reference bone without bone cancer; and
determining based on the comparison whether the cancer of the bone of the patient is present at the first location in the image or the second location in the image.

2. The method of claim 1, wherein the imaging is CT imaging.

3. The method of claim 1, wherein the imaging includes a first plurality of images in a first plane.

4. The method of claim 3, wherein the obtaining step, comparing step, and determining step is performed for each of the first plurality of images in the first plane.

5. The method of claim 4, wherein the imaging includes a second plurality of images in a second plane, and a third plurality of images in a third plane.

6. The method of claim 5, wherein the first plane, the second plane, and the third plane are different planes.

7. The method of claim 5, wherein the first plane is an axial plane, the second plane is a sagittal plane, and the third plane is a coronal plane.

8. The method of claim 6, wherein the obtaining step, comparing step, and determining step is performed for each of the second plurality of images in the second plane and for each of the third plurality of images in the third plane.

9. The method of claim 8, further comprising defining a three dimensional shape of the cancer based on the determining steps performed on each of the first, second, and third pluralities of images.

10. The method of claim 1, wherein the reference bone density ratio of interest is a reference ratio of a first reference density of a reference bone at a first reference location in a reference image to a second reference density of the reference bone at a second reference location in the reference image.

11. The method of claim 10, wherein the bone density ratio of interest is based on a plurality of reference bones of a reference population of reference patients.

12. The method of claim 11, wherein the first location and the second location are measured from an anatomical landmark.

13. The method of claim 12, wherein the first reference location and the second location are measured from a reference anatomical landmark.

14. The method of claim 13, wherein the first and second locations, and the first and second reference locations, are measured as percentile distances from the anatomical landmark and the reference anatomical landmark, respectively.

15. The method of claim 11, wherein the reference population comprises a group of individuals having a parameter in common with the patient.

16. The method of claim 15, wherein the parameter is selected from the group consisting of sex, age, and race.

17. The method of claim 1, wherein the first density of the bone is measured as a first value in Hounsfield units and the second density of the bone is measured as a second value in Hounsfield units.

18. The method of claim 1, wherein the first and second locations are both within a cortical shell of the bone.

19. The method of claim 18, wherein the first bone density represents a first maximum bone density at the first location.

20. The method of claim 19, wherein the second bone density represents a second maximum bone density at the second location.
